(19) United States
(12) Patent Application Publication
(10) Pub. No.: US 2024/0173302 A1
(43) Pub. Date: May 30, 2024

(54) TREATMENT OF INFLAMMATORY DISEASES

(71) Applicant: DIMERIX BIOSCIENCE PTY LTD, Fitzroy (AU)

(72) Inventors: Robert Pearson Shepherd, Upwey (AU); Nina Webster, Strathmore (AU)

(21) Appl. No.: 18/282,105

(22) PCT Filed: Mar. 18, 2022
(86) PCT No.: PCT/AU2022/050249

§ 371 Date: Sep. 14, 2023

(30) Foreign Application Priority Data
Mar. 23, 2021 (AU) 2021900862

(51) Int. Cl. A61K 31/422; A61K 31/194; A61K 31/4025; A61K 31/41; A61K 31/4178; A61K 31/4184; A61K 31/4245; A61K 31/505; A61K 31/506; A61K 31/519; A61K 38/12

(52) U.S. Cl. CPC A61K 31/422; A61K 31/194; A61K 31/4025; A61K 31/41; A61K 31/4178; A61K 31/4184; A61K 31/4245; A61K 31/505; A61K 31/506; A61K 31/519; A61K 38/12

(57) ABSTRACT

A pharmaceutical composition comprising at least one endothelin A receptor inhibitor or a pharmaceutically acceptable salt thereof and at least one chemokine receptor 2 (CCR2) inhibitor or a pharmaceutically acceptable salt thereof.

### TECHNICAL FIELD

The present invention relates to a combination therapy, comprising at least one chemokine receptor 2 pathway inhibitor and at least one endothelin A receptor inhibitor.

### BACKGROUND ART

Inflammatory disease pathologies (particularly those associated with the interaction between monocyte chemoattractant protein-1 (MCP-1) and C—C chemokine receptor type 2 (CCR2), and those associated with dysfunction of the endothelin system) have been associated with diseases such as uveitis, atherosclerosis, rheumatoid arthritis, multiple sclerosis, neuropathic pain, pruritis, Crohn's Disease, nephritis, organ allograft rejection, fibroid lung, renal insufficiency, diabetes and diabetic complications, diabetic nephropathy, diabetic retinopathy, diabetic retinitis, diabetic microangiopathy, tuberculosis, sarcoidosis, invasive staphylococcia, inflammation after cataract surgery, allergic rhinitis, allergic conjunctivitis, chronic urticaria, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, or stomach.

There is a need to provide new treatments for inflammatory disease pathologies including kidney, lung and heart disease; or at least the provision of alternative treatments to compliment the previously known treatments for inflammatory disease pathologies.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a pharmaceutical composition comprising:
a) at least one endothelin A receptor inhibitor or a pharmaceutically acceptable salt thereof; and
b) at least one chemokine receptor 2 (CCR2) inhibitor or a pharmaceutically acceptable salt thereof.

The present invention further provides a pharmaceutical composition comprising:
a) at least one endothelin A receptor inhibitor or a pharmaceutically acceptable salt thereof;
b) at least one chemokine receptor 2 (CCR2) inhibitor or a pharmaceutically acceptable salt thereof; and
c) at least one an angiotensin type 1 receptor (AT₁R) blocker or a pharmaceutically acceptable salt thereof.

Preferably the endothelin A receptor inhibitor is chosen from the group comprising: endothelin A receptor antagonists such as sitaxentan, ambrisentan, atrasentan, BQ-123, and zibotentan; dual endothelin A and B antagonists such as bosentan, macitentan, and tezosentan; and dual heterologous receptor antagonists such as sparsentan.

Preferably the chemokine receptor 2 (CCR2) inhibitor is chosen from the group comprising: repagermanium; propagermanium; CCX140; Cenicriviroc; PF-6309; PF-04178903; CCX872-B (CCX-872): MLN1202; and PF-04634817.

Preferably the angiotensin type 1 receptor (AT₁R) blocker is chosen from the group comprising: sparsentan, irbesartan, eprosartan, losartan, valsartan, telmisartan, candesartan, olmesartan, ZD-7115, fimasartan, azilsartan, DuP 753, EXP 3174, embusartan (BAY 10-6734), KRH-594, fonsartan (HR 720) and pratosartan (KT3-671).

The CCR2 pathway inhibitor, endothelin A receptor inhibitor and/or AT₁R blocker may be administered in the same dosage form or in separate dosage forms.

The CCR2 pathway inhibitor, endothelin A receptor inhibitor and/or AT₁R blocker may be administered concurrently or sequentially.

The invention also provides a method for the treatment, amelioration or prevention of a disease comprising administering to a subject a therapeutically effective amount of a combination of:
a) at least one endothelin A receptor inhibitor or a pharmaceutically acceptable salt thereof; and
b) at least one chemokine receptor 2 (CCR2) inhibitor or a pharmaceutically acceptable salt thereof.

The invention further provides a method for the treatment, amelioration or prevention of a disease comprising administering to a subject a therapeutically effective amount of a combination of:
a) at least one endothelin A receptor inhibitor or a pharmaceutically acceptable salt thereof;
b) at least one chemokine receptor 2 (CCR2) inhibitor or a pharmaceutically acceptable salt thereof and
c) at least one angiotensin type 1 receptor (AT1R) blocker or a pharmaceutically acceptable salt thereof.

Preferably the disease is a kidney disease, lung disease or cardiac disease. The kidney disease may be chosen from the list comprising: focal segmental glomerulosclerosis (FSGS; including primary FSGS and secondary FSGS), fibrotic disorders in the kidney, chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension. The lung disease may be an infectious or non-infectious inflammatory lung disease, preferably chosen from the list comprising: community acquired pneumonia, chronic obstructive pulmonary disease, asthma, bronchiectasis, bronchiolitis, bronchitis, emphysema, pleurisy or pulmonary fibrosis. The cardiac disease may be a coronary artery disease, preferably chosen from the list comprising: arthrosclerosis, pulmonary hypertension, ischaemic heart diseases, cardiomyopathies, and inflammatory heart disease.

The invention also contemplates the use of a pharmaceutical composition comprising:
a) at least one endothelin A receptor inhibitor or a pharmaceutically acceptable salt thereof; and
b) at least one chemokine receptor 2 (CCR2) inhibitor or a pharmaceutically acceptable salt thereof for the manufacture of a dosage form for the treatment, amelioration or prevention of a disease.

The invention further contemplates the use of a pharmaceutical composition comprising:
a) at least one endothelin A receptor inhibitor or a pharmaceutically acceptable salt thereof;
b) at least one chemokine receptor 2 (CCR2) inhibitor or a pharmaceutically acceptable salt thereof; and
c) at least one an angiotensin type 1 receptor (AT1R) blocker or a pharmaceutically acceptable salt thereof for the manufacture of a dosage form for the treatment, amelioration or prevention of a disease.

The present invention provides at least one endothelin A receptor inhibitor or a pharmaceutically acceptable salt thereof and at least one chemokine receptor 2 (CCR2) inhibitor or a pharmaceutically acceptable salt thereof, for use in a formulation for the treatment, amelioration or prevention of a disease.

The present invention provides at least one endothelin A receptor inhibitor or a pharmaceutically acceptable salt thereof, at least one chemokine receptor 2 (CCR2) inhibitor or a pharmaceutically acceptable salt thereof, and at least one angiotensin type 1 receptor (AT1R) blocker or a pharmaceutically acceptable salt thereof for use in a formulation for the treatment, amelioration or prevention of a disease.

The present invention provides:
at least one endothelin A receptor inhibitor for use in a formulation for the treatment, amelioration or prevention of a disease, wherein the at least one endothelin A receptor inhibitor is administered to the subject concurrently or sequentially with at least one CCR2 pathway inhibitor.
at least one CCR2 pathway inhibitor for use in a formulation for the treatment, amelioration or prevention of a disease wherein the at least one CCR2 pathway inhibitor is administered to the subject concurrently or sequentially with at least one endothelin A receptor inhibitor.
at least one endothelin A receptor inhibitor for use in a formulation for the treatment, amelioration or prevention of a disease, wherein the at least one endothelin A receptor inhibitor is administered to the subject concurrently or sequentially with at least one CCR2 pathway inhibitor and at least one angiotensin type 1 receptor blocker.
at least one CCR2 pathway inhibitor for use in a formulation for the treatment, amelioration or prevention of a disease wherein the at least one CCR2 pathway inhibitor is administered to the subject concurrently or sequentially with at least one endothelin A receptor inhibitor and at least one angiotensin type 1 receptor blocker.
at least one angiotensin type 1 receptor blocker for use in a formulation for the treatment, amelioration or prevention of a disease wherein the at least one CCR2 pathway inhibitor is administered to the subject concurrently or sequentially with at least one endothelin A receptor inhibitor and at least one CCR2 pathway inhibitor.

The present invention provides a kit for the treatment, amelioration or prevention of a disease, said kit comprising:
a) at least one chemokine receptor 2 (CCR2) pathway inhibitor or a pharmaceutically acceptable salt thereof;
b) at least one endothelin A receptor inhibitor or a pharmaceutically acceptable salt thereof; and
c) instructions for use.

The present invention provides a kit for the treatment, amelioration or prevention of a disease, said kit comprising:
a) at least one chemokine receptor 2 (CCR2) pathway inhibitor or a pharmaceutically acceptable salt thereof;
b) at least one endothelin A receptor inhibitor or a pharmaceutically acceptable salt thereof;
c) at least one angiotensin type 1 receptor (AT1R) blocker or a pharmaceutically acceptable salt thereof; and
d) instructions for use.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a graph of ETAR/Rluc8+Barr2/Venus+CCR2 which shows that when ETAR is tagged with Rluc8 and β-arrestin is tagged with Venus in the presence of unlabelled CCR2, β-arrestin is recruited to ETAR in the presence of ET-1 as expected, but also in the presence of CCL2/MCP-1 which confirms the presence of a complex between receptors. Presence of both ligands causes hyperactivation of β-arrestin recruitment, confirming the interaction of the heteromer.

FIG. 2 is a graph of ETAR/Rluc8+Barr2/Venus+CCR2 which shows β-arrestin recruitment to ETAR occurs when CCL2 is present, confirming the interaction of ETAR and unlabelled CCR2. Sparsentan is able to abrogate the hyperactivation when both CCL2/CMP-1 and ET-1 ligands are present but not cease recruitment of β-arrestin to ETAR.

FIG. 3 is a graph of ETAR/Rluc8+Barr2/Venus+CCR2 which shows the hyperactivation of ETAR and CCR2 heteromer is abrogated by the addition of either sparsentan or CCR2 inhibition. ETAR is able to recruit β-arrestin at a low magnitude when stimulated with ET-1 regardless of the presence of CCR2, and sparsentan inhibits this signalling independent of CCR2 activation. Complete inhibition of β-arrestin recruitment to ETAR only occurs when ET1 and CCL2/MCP-1 are present when CCR2 and ETAR are inhibited simultaneously.

FIG. 4 is a graph of CCR2/Rluc8+Barr2/Venus+HA-ETAR which shows in a different experimental orientation where CCR2 is tagged with Rluc8 and β-arrestin is tagged with Venus in the presence of unlabelled ETAR, β-arrestin is recruited to CCR2 in the presence of CCL2/MCP-1. Increased recruitment of β-arrestin is observed in the presence of both CCL2/MCP-1 and ET-1 ligands confirming the presence of an interaction between CCR2 and ETAR.

FIG. 5 is a graph of CCR2/Rluc8+Barr2/Venus+HA-ETAR which shows increased recruitment of β-arrestin is not observed in the presence of CCL2/MCP-1 and ET-1 when ETAR activation is blocked with sparsentan.

FIG. 6 is a graph of CCR2/Rluc8+Barr2/Venus+HA-ETAR which shows that there is no recruitment of β-arrestin to CCR2 in the presence of CCL2/MCP-1 when CCR2 is inhibited, regardless of the presence or absence of ETAR activation with ET-1.

### DESCRIPTION OF INVENTION

### Detailed Description of the Invention

Studies have shown that G protein-coupled receptors (GPCRs) may not only act as monomers but also as homo- and hetero-dimers and/or homo- and hetero-oligomers (also known as homomers and heteromers), which causes altered ligand binding, signalling and endocytosis (Rios et al. (2000) Pharmacol. Ther. 92:71-87). The effect of drugs acting as agonists or antagonists of a specific receptor may therefore depend on the binding partners of this receptor. It may be desirable to limit the effect of a drug to a cellular response mediated by a specific receptor dimer or oligomer.

### Composition

The present invention provides a pharmaceutical composition comprising:
a) at least one endothelin A receptor inhibitor or a pharmaceutically acceptable salt thereof; and
b) at least one chemokine receptor 2 (CCR2) inhibitor or a pharmaceutically acceptable salt thereof.

The present invention further provides a pharmaceutical composition comprising:
a) at least one endothelin A receptor inhibitor or a pharmaceutically acceptable salt thereof;
b) at least one chemokine receptor 2 (CCR2) inhibitor or a pharmaceutically acceptable salt thereof; and
c) at least one angiotensin type 1 receptor (AT₁R) blocker or a pharmaceutically acceptable salt thereof.

The pharmaceutical composition may further comprise a pharmaceutically acceptable carrier or excipient.

Without being held to any theory, it is believed that the chemokine receptor 2 and the endothelin A receptor form a hetero-dimer/-oligomer in some tissues. When the CCR2 and ETAR are in their hetero-dimer/-oligomer configuration, inhibition of both receptors at the same time causes a greater effect than inhibition of either receptor alone, or either receptor when the receptors are not in a heterodimeric configuration.

It is further believed that the chemokine receptor 2 and the angiotensin type 1 receptor form a hetero-dimer/-oligomer in some tissues. When the CCR2 and AT₁R are in their hetero-dimer/-oligomer configuration, inhibition of both receptors at the same time causes a greater effect than inhibition of either receptor alone, or either receptor when the receptors are not in a heterodimeric configuration.

The above finding allows specific and targeted interventions by the simultaneous inhibition of both elements of the hetero-dimer/-oligomer, which will result in a greater therapeutic effect in tissues where heterodimers form, but no greater therapeutic effect in tissues where the receptors are not in heterodimeric form.

### Chemokine Receptor 2 Pathway Inhibitor

C—C chemokine receptor type 2 (CCR2 or CD192) is a human G protein-coupled receptor. CCR2 is the primary receptor for monocyte chemoattractant protein-1 (MCP-1/CCL2).

The phrase “chemokine receptor 2 pathway” is to be understood to include any one of the pathways of the chemokine receptor 2 activated by its native ligands, including but not limited to those pathways that cause chemotactic migration, cell motility, extracellular-regulated kinase (ERK) phosphorylation, cAMP production, actin-recruitment, protein phosphorylation, nuclear protein localization, gene transcription or translation, G protein coupling, ß-arrestin recruitment or mediated signalling.

The phrase “chemokine receptor 2 pathway inhibitor” is intended to include any compound or agent which inhibits or partially inhibits any one of the pathways associated with the chemokine receptor 2. CCR2 pathway inhibitors includes compounds or agents which inhibit or partially inhibit activation of the chemokine receptor 2. The CCR2 pathway inhibitor may be a peptide, polypeptide or small chemical entity. For example, the CCR2 pathway inhibitor may be a protein, binding protein or antibody.

This may be through binding to the CCR2 ligand, monocyte chemoattractant protein-1 (MCP-1/CCL2), such that it can no longer bind to the chemokine receptor 2 (CCR2). In one embodiment the chemokine receptor 2 pathway inhibitor inhibits or partially inhibits the in vitro chemotactic migration of monocytes induced by MCP-1 by targeting MCP-1 directly. Such compounds include agents which bind MCP-1.

Alternatively, the chemokine receptor 2 pathway inhibitor may bind to the chemokine receptor 2 itself, blocking access or binding of the ligand. Such CCR2 pathway inhibitors may be a direct CCR2 antagonist, a direct CCR2 inverse agonist or a direct CCR2 negative allosteric modulator. A CCR2 antagonist would block the CCR2 and prevent binding of MCP-1; a CCR2 inverse agonist would bind to the CCR2 and decrease its activity below the basal level; and a CCR2 negative allosteric modulator would bind to the CCR2 and decrease its affinity to MCP-1 and/or its efficacy once MCP-1 is bound.

The phrase “chemokine receptor 2 pathway inhibitor” includes compounds or agents which inhibit or partially inhibit a component of the chemokine receptor 2 pathway other than the chemokine receptor 2 itself. For example, the inhibitor may inhibit or partially inhibit proteins that associate with chemokine receptor 2 or may inhibit compounds or pathway steps before and/or after the chemokine receptor itself. In such a case, preferably, the chemokine receptor 2 pathway inhibitors are selected from: an indirect CCR2 antagonist, an indirect CCR2 inverse agonist or an indirect CCR2 negative allosteric modulator.

The term “a component of the chemokine receptor 2 pathway other than the chemokine receptor 2 itself” as used herein, is to be understood to refer a component of the chemokine receptor 2 pathway wherein the component is itself not the chemokine receptor 2. The component of the chemokine receptor 2 pathway may interact directly with chemokine receptor 2. Alternatively, the component of the chemokine receptor 2 pathway may interact indirectly with chemokine receptor 2 by way of protein-protein interaction or complex formation. Alternatively, the component of the chemokine receptor 2 pathway may interact indirectly with chemokine receptor 2 by way of a signalling cascade. Preferably, the component is a protein such as, but not limited to, a transduction or signalling protein.

In one aspect, if the chemokine receptor 2 pathway inhibitor inhibits or partially inhibits a component of the pathway other than the chemokine receptor 2 itself, the inhibitor blocks MCP-1 induced migration and activation of monocytes and chemotactic migration through the targeting of glycosylphosphatidylinositol (GPI)-anchored proteins such as CD55, CD59 and CD16.

In another aspect, if the chemokine receptor 2 pathway inhibitor inhibits or partially inhibits a component of the pathway other than the chemokine receptor 2, the inhibited component is chosen from the list of CCR2 ligands comprising: CCL7 (MCP-3), CCL8, CCL13 (MCP-4), and CCL16 (hemofiltrate CC chemokine (HCC)-4). In one embodiment the chemokine receptor 2 pathway inhibitor inhibits or partially inhibits the in vitro chemotactic migration of monocytes induced by the above CCR2 ligands by targeting the ligands directly. Such compounds include agents which bind CCL7, CCL8, CCL13, and CCL16.

In another embodiment the chemokine receptor 2 pathway inhibitor inhibits or partially inhibits the in vitro chemotactic migration of monocytes induced by MCP-1 by targeting one or more GPI-anchored proteins selected from the group comprising CD55, CD59 and CD16. In such a case, the chemokine receptor 2 pathway inhibitor may stabilize the complexes CCR2/CD55 and/or CCR2/CD59 and/or CCR2/CD16.

Known inhibitors of the CCR2 pathway include: repagermanium or propagermanium; RS504393; RS102895; MLN-1202 (Millennium Pharmaceuticals); INCB3344, INCB3284 and INCB8696 (Incyte Pharmaceuticals); MK-0812 (Merck); CCX140 and CCX872-B (CCX-872); (ChemoCentryx); BMS-741672 (Bristol-Myers Squibb); and PF-04136309 (also known as PF-6309), PF-04178903 and PF-04634817 (Pfizer); and Cenicriviroc (TAK-652, TBR-652). Preferably, the chemokine receptor 2 pathway 2 inhibitor is chosen from the list: repagermanium; propagermanium; CCX140; PF-6309 and PF-04634817.

In one preferred embodiment the CCR2 pathway inhibitor is a direct antagonist of chemokine receptor 2, such as repagermanium, propagermanium or CCX140.

Propagermanium (3-oxygermylpropionic acid polymer), is a molecule that has been used as a therapeutic agent against chronic hepatitis, also has been shown to specifically inhibit in vitro chemotactic migration of monocytes by MCP-1 through a mechanism that seems to require glycosylphosphatidylinositol (GPI)-anchored proteins such as CD55, CD59 and CD16. Propagermanium is also known as 3-[(2-Carboxyethyl-oxogermyl)oxy-oxogermyl]propanoic acid, proxigermanium, Ge-132, bis (2-carboxyethylgermanium) sesquioxide (CEGS), 2-carboxyethylgermasesquioxane, SK-818, organic germanium, germanium sesquioxide, 3,3<′>-(1,3-dioxo-1,3-digermanoxanediyl) bispropionic acid, 3-oxygermylpropionic acid polymer, poly-fra/7s-(2-carboxyethyl) germasesquioxane, proxigermanium, repagermanium and Serocion™.

### Endothelin a Receptor Inhibitor

Endothelin receptor type A (also known as ETAR, ET_{A}, EDNRA, ET1 Receptor, ETA receptor, ETA, EDN1 and ET-AR), is a human G protein-coupled receptor.

The phrase “endothelin A receptor inhibitor” (also known as ETAR inhibitor) is intended to include any compound or agent which inhibits or partially inhibits any one of the pathways associated with the endothelin A receptor. The ETAR inhibitor is understood to include any compound or agent which inhibits or partially inhibits the activation of ETAR. The ETAR inhibitor may be a peptide, polypeptide or small chemical entity. For example, the ETAR inhibitor may be a protein, binding protein or antibody.

This may be through binding to the ETAR ligand, endothelin-1 (ET-1), such that it can no longer bind to the endothelin A receptor (ETAR). In one embodiment the ETAR inhibitor inhibits or partially inhibits the signaling induced by ET-1 by targeting ET-1 directly. Such compounds include agents which bind ET-1.

Alternatively, the ETAR inhibitor may bind to the ETAR itself, blocking access or binding of the ligand. Such ETAR inhibitors may be a direct ETAR antagonist, a direct ETAR inverse agonist or a direct ETAR negative allosteric modulator. An ETAR antagonist would block the ETAR and prevent binding of ET-1; an ETAR inverse agonist would bind to the ETAR and decrease its activity below the basal level; and an ETAR negative allosteric modulator would bind to the ETAR and decrease its affinity to ET-1 and/or its efficacy once ET-1 is bound.

Known inhibitors of the endothelin A receptor include: endothelin A receptor antagonists such as sitaxentan, ambrisentan (Volibris®, GalaxoSmithKline), atrasentan (AbbVie), BQ-123, zibotentan (ZD4054, AstraZeneca); dual endothelin A and B antagonists such as bosentan, macitentan, tezosentan; and dual heterologous receptor antagonists such as sparsentan.

As an example, the endothelin A receptor inhibitor may be sparsentan. Sparsentan, also known as 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide, is a combined selective antagonist of the angiotensin II type 1 receptor (AT₁R) and the ETA receptor.

### Angiotensin Type 1 Receptor

The angiotensin type 1 receptor (AT₁R, AT1R, angiotensin II receptor type 1) is a G protein-coupled receptor.

The phrase “angiotensin type 1 receptor blocker” (also referred to as AT₁R blocker or ARB) is intended to include any compound or agent which inhibits or partially inhibits any one of the pathways associated with the angiotensin type 1 receptor. The AT₁R blocker is understood to include any compound or agent which inhibits or partially inhibits the activation of AT₁R. The AT₁R blocker may be a peptide, polypeptide or small chemical entity. For example, the AT₁R blocker may be a protein, binding protein or antibody.

This may be through binding to the AT₁R ligands, for example angiotensin II (Ang II), such that it can no longer bind to AT₁R. In one embodiment the AT₁R blocker inhibits or partially inhibits the signaling induced by the binding of the AT₁R ligand to the AT₁R, by targeting the AT₁R ligand directly. Such compounds include agents which bind the AT₁R ligand, for example agents which bind angiotensin II (Ang II).

Alternatively, the AT₁R blocker may bind to the AT₁R itself, blocking access or binding of the ligand. Such AT₁R blocker may be a direct AT₁R antagonist, a direct AT₁R inverse agonist or a direct AT₁R negative allosteric modulator. An AT₁R antagonist would block the AT₁R and prevent binding of the AT₁R ligand; an AT₁R inverse agonist would bind to the AT₁R and decrease its activity below the basal level; and an AT₁R negative allosteric modulator would bind to the AT₁R and decrease its affinity to the AT₁R ligand and/or its efficacy once the AT₁R ligand is bound.

For example, the AT₁R blocker may be selected from the group comprising: sparsentan, irbesartan, eprosartan, losartan, valsartan, telmisartan, candesartan, olmesartan, ZD-7115, fimasartan, azilsartan, DuP 753 (2-n-Butyl-4-chloro-5-hydroxy-methyl-1-[(2′-(1H)-tetrazol-5-yl)biphenyl-4-yl)methyl]imidazol potassium salt), EXP 3174, embusartan (BAY 10-6734), KRH-594, fonsartan (HR 720) and pratosartan (KT3-671).

Taylor et al (2011) reviews AT₁R blockers and noted that “[t]here are no major differences in efficacy or other clinical characteristics among older drugs in this class, although some of the newer agents may more effectively reduce blood pressure than the older agents”. The differences in characteristics appear to be related to interaction with other pathways and not the core angiotensin type 1 receptor blockage. It is noted that although “[t]here are some pharmacological differences among the ARBs, and some drugs in this class have special effects independent of angiotensin II receptor blockade, yet there is currently no strong evidence that these translate into unique protection against target organ damage from hypertension”. Thus, in respect of their effect on AT₁R blockage, AT₁R blockers are functionally interchangeable despite structural differences.

AT₁R blockers first become commercially available in the 1990's. Despite the extended period of use of this class of drugs, physicians treat these as a group which are largely substitutable with each other. While there are some specific subtleties in the exact pharmacology of some of the group, these appear not to be related to their angiotensin type 1 receptor activity. Furthermore, all pharmacological products will behave subtly differently in some patients, and yet some sub-group variability does not prevent them from being generally useful in therapy (Taylor et al., 2011).

As an example, the angiotensin receptor inhibitor may be irbesartan. Irbesartan is an angiotensin type 1 receptor antagonist also known as 2-butyl-3-({4-[2-(2H-1,2,3,4-tetrazol-5-yl)phenyl]phenyl}methyl)-1,3-diazaspiro[4.4]non-1-en-4-one. The angiotensin receptor inhibitor may be sparsentan. Sparsentan, also known as 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide, is a combined selective antagonist of the angiotensin II type 1 receptor (AT₁R) and the ETA receptor.

### Method of Treatment

The invention also provides a method for the treatment, amelioration or prevention of a disease comprising administering to a subject a therapeutically effective amount of a combination of:
a) at least one endothelin A receptor inhibitor or a pharmaceutically acceptable salt thereof; and
b) at least one chemokine receptor 2 (CCR2) inhibitor or a pharmaceutically acceptable salt thereof.

The invention further provides a method for the treatment, amelioration or prevention of a disease comprising administering to a subject a therapeutically effective amount of a combination of:
a) at least one endothelin A receptor inhibitor or a pharmaceutically acceptable salt thereof;
b) at least one chemokine receptor 2 (CCR2) inhibitor or a pharmaceutically acceptable salt thereof and
c) at least one angiotensin type 1 receptor (AT₁R) blocker or a pharmaceutically acceptable salt thereof.

The CCR2 pathway inhibitor and the endothelin A receptor inhibitor may be administered: in the same dosage form or in separate dosage forms. The CCR2 pathway inhibitor and the endothelin A receptor inhibitor may be administered: concurrently or sequentially.

If the method of treatment, amelioration or prevention further comprises at least one angiotensin type 1 receptor (AT₁R) blocker, the AT₁R blocker may be administered: in the same dosage form as the CCR2 pathway inhibitor and/or the ETAR inhibitor or in separate dosage forms. The angiotensin type 1 receptor (AT₁R) blocker, CCR2 pathway inhibitor and the endothelin A receptor inhibitor may be administered: concurrently or sequentially.

The endothelin A receptor inhibitor, CCR2 pathway inhibitor and/or the AT₁R blocker may be pharmaceutically acceptable salts of the endothelin A receptor inhibitor, CCR2 pathway inhibitor and/or the AT₁R blocker.

One component of the treatment of the present invention may already be being administered to a subject, for example as standard of care treatment. In such a case, the second (and third if present) component of the treatment of the present invention is administered as a second (and third) component in therapy to provide the therapy of the present invention.

While not intending to be restricted to any particular mode of action, in one preferred embodiment the CCR2 pathway inhibitor has a greater affinity and/or potency and/or efficacy when interacting with the CCR2 or modulating its downstream pathways when the CCR2 is associated with the endothelin A receptor. For example, the CCR2 and the endothelin A receptor may be associated as a CCR2/ETAR hetero-dimer/-oligomer. In a further preferred embodiment, when the CCR2 pathway inhibitor is administered to a subject concurrently or sequentially with an endothelin A receptor inhibitor, the combined affinity, potency and/or efficacy is greater than compared to the affinity, potency and/or efficacy that would have been achieved when the CCR2 pathway inhibitor is not administered (whether concurrently or sequentially) with the endothelin A receptor inhibitor. In an even further preferred embodiment, a synergistic effect (as measured by affinity, potency and/or efficacy) is achieved when the CCR2 pathway inhibitor is administered to a subject (whether concurrently or sequentially) with the endothelin A receptor inhibitor.

While not intending to be restricted to any particular mode of action, in one preferred embodiment the endothelin A receptor inhibitor has a greater affinity and/or potency and/or efficacy when interacting with the endothelin A receptor when the endothelin A receptor is associated with the CCR2. For example, the CCR2 and the endothelin A receptor may be associated as a CCR2/ETAR hetero-dimer/-oligomer. In a further preferred embodiment, when the endothelin A receptor inhibitor is administered to a subject concurrently or sequentially with a CCR2 pathway inhibitor, the combined affinity, potency and/or efficacy is greater than compared to the affinity, potency and/or efficacy that would have been achieved when the endothelin A receptor inhibitor is not administered (whether concurrently or sequentially) with the CCR2 pathway inhibitor. In an even further preferred embodiment, a synergistic effect (as measured by affinity, potency and/or efficacy) is achieved when the endothelin A receptor inhibitor is administered to a subject (whether concurrently or sequentially) with a CCR2 pathway inhibitor.

While not intending to be restricted to any particular mode of action, in one preferred embodiment the CCR2 pathway inhibitor has a greater affinity and/or potency and/or efficacy when interacting with the CCR2 or modulating its downstream pathways when the CCR2 is associated with the angiotensin receptor. For example, the CCR2 and the angiotensin receptor may be associated as a CCR2/AT₁R hetero-dimer/-oligomer. In a further preferred embodiment, when the CCR2 pathway inhibitor is administered to a subject concurrently or sequentially with an AT₁R blocker, the combined affinity, potency and/or efficacy is greater than compared to the affinity, potency and/or efficacy that would have been achieved when the CCR2 pathway inhibitor is not administered (whether concurrently or sequentially) with the AT₁R blocker. In an even further preferred embodiment, a synergistic effect (as measured by affinity, potency and/or efficacy) is achieved when the CCR2 pathway inhibitor is administered to a subject (whether concurrently or sequentially) with an AT₁R blocker.

While not intending to be restricted to any particular mode of action, in one preferred embodiment the AT₁R blocker has a greater affinity and/or potency and/or efficacy when interacting with the angiotensin receptor when the angiotensin receptor is associated with the CCR2. For example, the CCR2 and the angiotensin receptor may be associated as a CCR2/AT₁R hetero-dimer/-oligomer. In a further preferred embodiment, when the AT₁R blocker is administered to a subject concurrently or sequentially with a CCR2 pathway inhibitor, the combined affinity, potency and/or efficacy is greater than compared to the affinity, potency and/or efficacy that would have been achieved when the AT₁R blocker is not administered (whether concurrently or sequentially) with the CCR2 pathway inhibitor. In an even further preferred embodiment, a synergistic effect (as measured by affinity, potency and/or efficacy) is achieved when the AT₁R blocker is administered to a subject (whether concurrently or sequentially) with a CCR2 pathway inhibitor.

As the CCR2/ETAR hetero-dimer/-oligomer has greater affinity, potency and/or efficacy than each element alone when simultaneously activated by their cognate ligands, and the CCR2/AT₁R hetero-dimer/-oligomer has greater affinity, potency and/or efficacy than each element alone when activated by their cognate ligands, it is anticipated that inhibition of the two sets of hetero-dimer/-oligomers concurrently will result in high levels of therapeutic advantage.

The subject to be treated is preferably a mammal, including a human mammal.

### Condition or Disease

Preferably the CCR2 pathway inhibitor, endothelin A receptor inhibitor and/or the AT₁R blocker are for use in the treatment, amelioration or prevention of a disease chosen from the list comprising: lung disease, kidney disease, cardiovascular disease including, (acute and chronic) congestive heart failure, left ventricular dysfunction and hypertrophic cardiomyopathy, diabetic cardiac myopathy, supraventricular and ventricular arrhythmias, atrial fibrillation or atrial flutter, myocardial infarction and its sequelae, atherosclerosis, angina (whether unstable or stable), heart failure, angina pectoris, diabetes, secondary aldosteronism, primary and secondary pulmonary hyperaldosteronism, primary and pulmonary hypertension, diabetic retinopathy, macular degeneration, ocular disorders, insulin resistance, the management of other vascular disorders, such as migraine, Raynaud's disease, luminal hyperplasia, cognitive dysfunction (such as Alzheimer's), stroke, hyperkalemia, preeclampsia, sarcoidosis, ischemia and reperfusion injury, atherogenesis, chronic obstructive pulmonary disease, asthma and allergy renal disease, rheumatoid arthritis, neuropathic pain, and pruritis.

The endothelin A receptor inhibitor, CCR2 pathway inhibitor and/or the AT₁R blocker may be used in the treatment, amelioration or prevention of kidney disease, more particularly a disease chosen from the list comprising: focal segmental glomerulosclerosis (FSGS; including primary FSGS and secondary FSGS), fibrotic disorders in the kidney, chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension.

The endothelin A receptor inhibitor, CCR2 pathway inhibitor and/or the AT₁R blocker may be used in the treatment, amelioration or prevention of a lung disease, preferably an infectious or noninfectious inflammatory lung disease. Preferably the infectious or non-infectious inflammatory lung disease is chosen from the list comprising: community acquired pneumonia, chronic obstructive pulmonary disease, asthma, bronchiectasis, bronchiolitis, bronchitis, emphysema, pleurisy or pulmonary fibrosis.

The endothelin A receptor inhibitor, CCR2 pathway inhibitor and/or the AT₁R blocker may be used in the treatment, amelioration or prevention of a cardiac disease. The cardiac disease may be chosen from the list comprising: coronary artery diseases including arthrosclerosis, pulmonary hypertension, ischaemic heart diseases, cardiomyopathies, and inflammatory heart disease.

The term “prevention” includes administration of the compositions of the present invention to subjects who have symptoms that indicate they are at risk of developing a disease, but may not, at the time of treatment, be diagnosed with the disease. For example, the subject may have diabetes, but may not yet have been diagnosed with diabetes-related kidney disease.

The term “amelioration” includes administration of the compositions of the present invention to subjects who have had the disease and are at risk of developing post-disease conditions such as fibrosis and persistent inflammation. The post-disease conditions may be due to the disease itself or may be due to medical interventions such as ventilators used during the acute phase of a lung disease.

### Measurement of Inhibition

The phrase “endothelin A receptor inhibitor” is understood to include any compound or agent which inhibits or partially inhibits the activation of the endothelin A receptor.

The phrase “angiotensin type 1 receptor blocker” is understood to include any compound or agent which inhibits or partially inhibits the activation of AT₁R.

The phrase “chemokine receptor 2 pathway inhibitor” is intended to include any compound or agent which inhibits or partially inhibits any one of the pathways associated with the chemokine receptor 2.

The term “inhibits” as used herein, means a reduction below detectable limits when compared to a reference. The phrase includes blocking, retarding, or impeding an action to prevent an undesirable result.

The term “partially inhibits” as used herein, means any reduction within detectable limits when compared to a reference. The phrase includes blocking, retarding, or impeding an action to prevent an undesirable result.

The inhibition or partial inhibition of the CCR2 pathway, endothelin A receptor and/or the AT₁R caused by (i) the CCR2 pathway inhibitor and the endothelin A receptor inhibitor, or (ii) the CCR2 pathway inhibitor, endothelin A receptor inhibitor and the AT₁R blocker, may be measured using the in vitro methods set out herein, and include but are not limited to, biochemical or cellular assays for the assessment of in vitro chemotactic migration of CCR2-expressing neutrophils and other cells such as are known in the art, as well as measurement of inositol phosphate production, extracellular-regulated kinase (ERK) phosphorylation, CAMP production, actin-recruitment, protein phosphorylation, nuclear protein localization, gene transcription, label-free technologies (such as using impedance, light refraction or charge redistribution), G protein coupling using proximity reporter systems or other approaches, β-arrestin recruitment or mediated signalling, transcription factor-based reporter systems, microscopy visualization using fluorescent labels, use of antibodies to assess receptor cellular localization (such as enzyme-linked immunosorbent assays) and fluorescence activated cell sorting.

The inhibition or partial inhibition of the CCR2 pathway endothelin A receptor and/or the AT₁R caused by (i) the CCR2 pathway inhibitor and the endothelin A receptor inhibitor, or (ii) the CCR2 pathway inhibitor, endothelin A receptor inhibitor and the AT₁R blocker, may be measured using the in vivo methods set out herein.

For example if the disease is a kidney disease, the measurements include but are not limited to, serial measurements of renal function made by the measurement of plasma creatinine and urea such as by way of an autoanalyser; the measurement of proteinuria, the measurement of albuminuria; and GFR or estimated GFR; the assessment of endpoints such as renal and/or cardiac and/or ocular structure, by way of, for example, light microscopy (LM) for the assessment of glomerular and cardiac hypertrophy, glomerulosclerosis and/or fibrosis and/or podocyte change and/or; immunohistochemistry to measure the extent of matrix deposition and modulation of profibrotic growth factors and their activity; assessment of systolic blood pressure, modulation of insulin fasting plasma glucose, modulation of Hemoglobin A1c; and molecular biological techniques to assess renal and cardiac and ocular structure according to conventional assays such as known in the art. Inhibition or partial inhibition may be indicated by a qualitative improvement in renal and/or cardiac and/or ocular structure as measured by one or more of the above mentioned endpoints, or other patient benefit endpoints such as quality of life improvement or renal or overall survival.

Alternatively, if the disease is a lung disease, the measurements include but are not limited to, measurement of cellular and cytokine content of lung exudate, measurement of lung function including physical capacity of lung function using spirometry-based tests, or lung functional outputs measured using measurement blood gas or other biochemical measures, or improvement in functional benefit including clinical benefit measured by survival or quantitative methods such as walk tests or qualitative methods such as patient-reported outcome assessment. Inhibition or partial inhibition may be indicated by a qualitative improvement in lung structure as measured by one or more of the above-mentioned endpoints.

In another embodiment, the total efficacy of the pharmaceutical formulation is greater when compared to the efficacies of the endothelin A receptor inhibitor, AT₁R blocker or the CCR2 pathway inhibitor when each component is administered without any administration of the other components. Thus, the combined formulation may be administered in a single dose, including at sub-therapeutic doses, or less often, than any of the components might be administered as single compounds.

Preferably, the total efficacy of the pharmaceutical formulation is greater when compared to the sum of the efficacies of the endothelin A receptor inhibitor, AT₁R blocker and/or the CCR2 pathway inhibitor when any component is administered without administration of the other components. More preferably, a synergistic effect in efficacy is observed when the endothelin A receptor inhibitor, AT₁R blocker and/or the CCR2 pathway inhibitor are administered concurrently or sequentially.

Alternatively, the total efficacy of the pharmaceutical formulation is equal to the sum of the efficacies of the endothelin A receptor inhibitor, AT₁R blocker and/or the CCR2 pathway inhibitor when any component is administered without any administration of the other components. As a further preferred embodiment of this alternative, an additive effect in efficacy is observed when the endothelin A receptor inhibitor, AT₁R blocker and/or the CCR2 pathway inhibitor are administered concurrently or sequentially.

In a further alternative, the total efficacy of the pharmaceutical formulation is less than the sum of the efficacies of the endothelin A receptor inhibitor, AT₁R blocker and/or the CCR2 pathway inhibitor when any component is administered without administration of the other components. In a further embodiment, while the combined efficacy is less than the sum of the efficacies of the endothelin A receptor inhibitor, AT₁R blocker and/or the CCR2 pathway inhibitor when each component is administered without any administration of the other components, the treatment provides greater efficacy compared to a single treatment of endothelin A receptor inhibitor, AT₁R blocker or the CCR2 pathway inhibitor administered alone.

Preferably the components are administered concurrently at the same time (for example as two or three tablets taken together, or as a single tablet, formulated with each component) or sequentially (for example one or two tablets taken after another tablet). The doses of each component may be taken together (concurrently), or sequentially and taken within seconds, minutes, days, weeks or months of each other.

### Delivery

The dosage form provided by the present invention may further comprise a vial, cartridge, container, tablet or capsule comprising the pharmaceutical formulation of the invention together with dosage instructions for the administration of the dosage form(s) to a subject for the treatment, amelioration or prevention of a disease.

The amount of each active ingredient which may be combined with the carrier materials to produce a single dosage will vary, depending upon the host to be treated and the particular mode of administration. For example, a formulation intended for oral administration to humans may contain about 0.5 mg to 1 g of each active compound with an appropriate and convenient amount of carrier material, which may vary from about 5 to 95% w/w of the total formulation. Dosage unit forms will generally contain between from about 0.5 mg to 500 mg of active ingredient(s).

Preferably, the CCR2 pathway inhibitor is provided at between 0.5 mg to 2000 mg per day, provided in one or more doses. Even more preferably the CCR2 pathway inhibitor is provided at a dose of between 0.5 mg to 50 mg per day, provided in one or more doses.

Preferably, the endothelin A receptor inhibitor is provided at between 0.5 mg to 2000 mg per day, provided in one or more doses. Even more preferably the endothelin A receptor inhibitor is provided at a dose of between 0.5 mg to 300 mg per day, provided in one or more doses. For example, the ETAR inhibitor is sparsentan and is administered at a dose of 75, 150 or 300 mg per day, provided in one or more doses. Other exemplary doses include: ambrisentan dosed at 5 mg or 10 mg per day, provided in one or more doses; bosentan dosed at 62.5 mg or 125 mg per day, provided in one or more doses; or macitentan dosed at 10 mg per day, provided in one or more doses.

Preferably, the AT₁R blocker is provided at between 50 mg to 500 mg per day, provided in one or more doses. Even more preferably, the AT₁R blocker is provided at between 75 mg to 300 mg per day. For example, the AT₁R blocker is irbesartan and is administered at a dose of 75, 150 or 300 mg per day, provided in one or more doses. For example, the AT₁R blocker is sparsentan and is administered at a dose of 75, 150 or 300 mg per day, provided in one or more doses.

The dose of each active agent may be provided in either a single dosage form, two separate dosage forms or three separate dosage forms. The actives may be provided as about 0.5 mg to 2 g of the CCR2 pathway inhibitor, about 0.5 mg to 2 g of the ETAR inhibitor and/or about 50 mg to 500 mg of the AT₁R blocker per day. The dose of the actives may be provided in either a single dosage form, two separate dosage forms or three separate dosage forms.

It will be understood, however, that the specific dose level for any particular subject will depend upon a variety of factors including the activity of the specific compound employed, the age, body weight, general health, sex, diet, time of administration, route of administration, rate of excretion, combinations of drugs, and the severity of the particular condition or disease undergoing therapy.

Formulations of the invention, in various aspects, may be administered by injection, or prepared for oral, pulmonary, nasal or for any other form of administration. Preferably the formulations are administered, for example, orally or via gastric feeding tube, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration.

The mode of administration is in one aspect at least suitable for the form in which the formulation has been prepared. The mode of administration for the most effective response may be determined empirically and the means of administration described below are given as examples, and do not limit the method of delivery of the formulation of the present invention in any way. All the formulations provided are commonly used in the pharmaceutical industry and are commonly known to suitably qualified practitioners.

### Injectable Dosage Forms

The formulations of the invention in certain aspects may include pharmaceutically acceptable non-toxic excipients and carriers and administered by any parenteral techniques such as subcutaneous, intravenous and intraperitoneal injections. In addition, the formulations may optionally contain one or more adjuvants. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

The pharmaceutical forms suitable for injectable use optionally include sterile aqueous solutions (where water-soluble) or dispersions and sterile powders for the extemporaneous preparation of sterile injectable solutions or dispersion. Alternatively, the compounds of the invention are, in certain aspects encapsulated in liposomes and delivered in injectable solutions to assist their transport across cell membrane. Alternatively, or in addition, such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by a coating such as lecithin; by the maintenance of the required particle size in the case of dispersion; and by the use of surfactants.

The invention also provides prolonged absorption of an injectable sustained release pharmaceutical formulation comprising a therapeutically effective pharmaceutical formulation according to the invention and a release retardant. The release retardant may be, for example, aluminium mono-stearate and gelatine.

Sterile injectable solutions are prepared by incorporating the active compounds in the required amount in an appropriate solvent with one or more of the other ingredients enumerated above, as required, followed by filtered sterilisation. Generally, dispersions are prepared by incorporating the various sterilised active ingredient into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the active ingredient plus any additional desired ingredient from previously sterile-filtered solution thereof.

### Oral Dosage Forms

Contemplated for use herein are oral dosage forms, which are described generally in Martin, Remington's Pharmaceutical Sciences, 18th Ed. (1990 Mack Publishing Co. Easton PA 18042) at Chapter 89, which is herein incorporated by reference. Solid dosage forms include tablets, capsules, pills, troches or lozenges, cachets, liquids, suspensions or pellets. Also, liposomal or proteinoid encapsulation may be used to formulate the present formulations (as, for example, proteinoid microspheres reported in U.S. Pat. No. 4,925,673). Liposomal encapsulation may be used, and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the formulation will include the compounds described as part of the invention (or a chemically modified form thereof), and inert ingredients which allow for protection against the stomach environment, and release of the biologically active material in the intestine.

The oral dosage form of the present invention may be a liquid, suspension or other appropriate dosage form that is delivered directly into the feeding tube of a patient who is in a coma and/or on a respirator and is unable to swallow a tablet etc.

For the CCR2 pathway inhibitor, endothelin A receptor inhibitor or AT₁R blocker of the invention, the location of release may be the stomach, the small intestine (the duodenum, the jejunum, or the ileum), or the large intestine. One skilled in the art has available formulations that will not dissolve in the stomach yet will release the material in the duodenum or elsewhere in the intestine. In one aspect, the release will avoid the deleterious effects of the stomach environment, either by protection of the formulation or by release of the compounds beyond the stomach environment, such as in the duodenum or elsewhere in the intestine.

A coating or mixture of coatings can also be used on tablets, which are not intended for protection against the stomach. This includes without limitation sugar coatings, or coatings that make the tablet easier to swallow. Exemplary capsules consist of a hard shell (such as gelatine) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical formulation has passed from the stomach and through until and after the pharmaceutical formulation reaches the intestine. The phrase “sustained release” also means delayed release wherein release of the therapeutic compound is not immediately initiated upon the pharmaceutical formulation reaching the stomach but rather is delayed for a period of time, for example, until when the pharmaceutical formulation reaches the intestine. Upon reaching the intestine, the increase in pH may then trigger release of the therapeutic compound from the pharmaceutical formulation.

Though term “release retardant” is used herein, means a substance that reduces the rate of release of a therapeutic compound from a pharmaceutical formulation when orally ingested. The release retardant may be a polymer or a non-polymer. The release retardant may be used according to any one of several sustained release systems including, for example, a diffusion system, a dissolution system and/or an osmotic system.

In certain aspects, the therapeutic is included in the formulation as fine multi-particulates in the form of granules or pellets of particle size about 1 mm. The formulation of the material for capsule administration is, in certain aspects, a powder, lightly compressed plugs or even as tablets. In one aspect, the therapeutic could be prepared by compression.

Colourants and flavouring agents are optionally all included. For example, compounds may be formulated (such as, and without limitation, by liposome or microsphere encapsulation) and then further contained within an edible product, such as a refrigerated beverage containing colorants and flavouring agents.

The volume of the therapeutics, in one aspect, diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the therapeutic compounds together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the formulation of the therapeutic to prevent sticking during the formulation process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during formulation and to aid rearrangement during compression might be optionally added. The glidants may include without limitation starch, talc, pyrogenic silica and hydrated silicoaluminate.

To aid dissolution of the therapeutic into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the formulation of the compounds either alone or as a mixture in different ratios.

Additives that potentially enhance uptake of the compounds are for instance and without limitation the fatty acids oleic acid, linoleic acid and linolenic acid.

Controlled release formulations may be desirable. These formulations are also contemplated. In certain aspects, the compounds could be incorporated into an inert matrix that permits release by either diffusion or leaching mechanisms i.e., gums. In some aspects, slowly degenerating matrices may also be incorporated into the formulation. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example and without limitation, in a pan coater or in a fluidized bed or by compression coating.

### Pulmonary and Nasal Dosage Forms

Also contemplated herein is pulmonary delivery of the formulations of the invention. In these aspects, the CCR2 pathway inhibitor, endothelin A receptor inhibitor, or the AT₁R blocker may be delivered to the lungs of a subject while inhaling and traverses across the lung epithelial lining to the blood stream.

Contemplated for use in the practice of this invention are a wide range of mechanical devices designed for pulmonary delivery of therapeutic products, including but not limited to nebulizers, metered-dose inhalers, and powder inhalers, all of which are familiar to those skilled in the art.

Some specific examples of commercially available devices suitable for the practice of this invention are, for example and without limitation, the Ultravent nebulizer, manufactured by Mallinckrodt, Inc., St. Louis, Missouri; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Massachusetts.

All such devices require the use of formulations suitable for the dispensing of the compounds. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise the compounds suspended in water. The formulation may also include, in one aspect, a buffer and a simple sugar (e.g., for protein stabilization and regulation of osmotic pressure). In one embodiment, the nebulizer formulation may also contain a surfactant, to reduce or prevent surface induced aggregation of the compounds caused by atomization of the solution in forming the aerosol.

Formulations for use with a metered dose inhaler device will generally comprise, in one aspect a finely divided powder containing the compounds suspended in a propellant with the aid of a surfactant. The propellant may be any conventional material employed for this purpose, such as and without limitation, a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include, without limitation sorbitan trioleate and soya lecithin. Oleic acid may also be useful as a surfactant in certain aspects.

Formulations for dispensing from a powder inhaler device will comprise a finely divided dry powder containing the compound and may also include a bulking agent, such as and without limitation lactose, sorbitol, sucrose, or mannitol in amounts which facilitate dispersal of the powder from the device, e.g., 50 to 90% by weight of the formulation. In certain embodiments, the compound(s) is/are prepared in particulate form with an average particle size of less than 10 microns, most preferably 0.5 to 5 microns, for most effective delivery to the distal lung.

Nasal delivery of the compounds is also contemplated. Nasal delivery allows the passage of the protein to the blood stream directly after administering the therapeutic product to the nose, without the necessity for deposition of the product in the lung. Formulations for nasal delivery include those with, for example and without limitation, dextran or cyclodextran.

### Dosing Schedule

It will be appreciated that in certain aspects, the formulations of the invention may be given as a single dose schedule, or preferably, in a multiple dose schedule. A multiple dose schedule is one in which a primary course of delivery may be with 1 to 10 separate doses, is optionally followed by other doses given at subsequent time intervals required to maintain or reinforce the treatment. The dosage regimen will also, at least in part, be determined by the needs of the individual and the judgement of the practitioner.

The invention thus provides a tablet comprising the pharmaceutical formulation of the invention; a capsule comprising the pharmaceutical formulation of the invention, an injectable suspension comprising the pharmaceutical formulation of the invention, and a formulation for pulmonary delivery comprising the pharmaceutical formulation of the invention. The endothelin A receptor inhibitor, AT₁R blocker and/or the CCR2 pathway inhibitor may be delivered in the same formulation or may be delivered in separate formulations.

The endothelin A receptor inhibitor, AT₁R blocker and/or the CCR2 pathway inhibitor may be in the same dosage form or may be in separate dosage forms. The subject being administered the endothelin A receptor inhibitor, AT₁R blocker and/or the CCR2 pathway inhibitor may be already receiving one or more of the active agents and may, in accordance with the present invention, be administered the other component(s) of the treatment of the present invention. The treatment of the present invention may comprise the administration of only a CCR2 pathway inhibitor and an endothelin A receptor inhibitor, or may comprise administration of a CCR2 pathway inhibitor, an endothelin A receptor inhibitor and an AT₁R blocker.

### Excipients

The CCR2 pathway inhibitor, ETAR inhibitor and AT₁R blocker may be pharmaceutically acceptable salts of the respective active agent. Pharmaceutically and veterinary acceptable salts include salts which retain the biological effectiveness and properties of the compounds of the present disclosure and which are not biologically or otherwise undesirable. In many cases, the compounds disclosed herein are capable of forming acid and/or base salts by virtue of the presence of amino and/or carboxyl groups or groups similar thereto. Acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as by way of example only, alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically and veterinary acceptable acid addition salts may be prepared from inorganic and organic acids. The inorganic acids that can be used include, by way of example only, hydrochloric acid, hydrobromic acid, sulfuric acid, nitric acid, phosphoric acid, and the like. The organic acids that can be used include, by way of example only, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The pharmaceutically or veterinary acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences. 17th ed., Mack Publishing Company, Easton, Pa. (1985), p. 1418, the disclosure of which is hereby incorporated by reference. Examples of such acceptable salts are the iodide, acetate, phenyl acetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, γ-hydroxybutyrate, ß-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, merhanesulfonate, naphthalene-l-sulfonate, naphthalene-2-sulfonate, p-toluenesulfonate, xylenesulfonate, tartarate, and the like.

### Use

The invention also contemplates the use of a pharmaceutical composition comprising:
a) at least one endothelin A receptor inhibitor or a pharmaceutically acceptable salt thereof; and
b) at least one chemokine receptor 2 (CCR2) inhibitor or a pharmaceutically acceptable salt thereof
   for the manufacture of a dosage form for the treatment, amelioration or prevention of a disease.

The invention further contemplates the use of a pharmaceutical composition comprising:
a) at least one endothelin A receptor inhibitor or a pharmaceutically acceptable salt thereof;
b) at least one chemokine receptor 2 (CCR2) inhibitor or a pharmaceutically acceptable salt thereof; and
c) at least one an angiotensin type 1 receptor (AT₁R) blocker or a pharmaceutically acceptable salt thereof
   for the manufacture of a dosage form for the treatment, amelioration or prevention of a disease.

The present invention provides at least one endothelin A receptor inhibitor or a pharmaceutically acceptable salt thereof and at least one chemokine receptor 2 (CCR2) inhibitor or a pharmaceutically acceptable salt thereof, for use in a formulation for the treatment, amelioration or prevention of a disease.

The present invention provides at least one endothelin A receptor inhibitor or a pharmaceutically acceptable salt thereof, at least one chemokine receptor 2 (CCR2) inhibitor or a pharmaceutically acceptable salt thereof, and at least one angiotensin type 1 receptor (AT₁R) blocker or a pharmaceutically acceptable salt thereof for use in a formulation for the treatment, amelioration or prevention of a disease.

The present invention provides:
at least one endothelin A receptor inhibitor for use in a formulation for the treatment, amelioration or prevention of a disease, wherein the at least one endothelin A receptor inhibitor is administered to the subject concurrently or sequentially with at least one CCR2 pathway inhibitor.
at least one CCR2 pathway inhibitor for use in a formulation for the treatment, amelioration or prevention of a disease wherein the at least one CCR2 pathway inhibitor is administered to the subject concurrently or sequentially with at least one endothelin A receptor inhibitor.
at least one endothelin A receptor inhibitor for use in a formulation for the treatment, amelioration or prevention of a disease, wherein the at least one endothelin A receptor inhibitor is administered to the subject concurrently or sequentially with at least one CCR2 pathway inhibitor and at least one angiotensin type 1 receptor blocker.
at least one CCR2 pathway inhibitor for use in a formulation for the treatment, amelioration or prevention of a disease wherein the at least one CCR2 pathway inhibitor is administered to the subject concurrently or sequentially with at least one endothelin A receptor inhibitor and at least one angiotensin type 1 receptor blocker.
at least one angiotensin type 1 receptor blocker for use in a formulation for the treatment, amelioration or prevention of a disease wherein the at least one CCR2 pathway inhibitor is administered to the subject concurrently or sequentially with at least one endothelin A receptor inhibitor and at least one CCR2 pathway inhibitor.

Preferably the disease is a kidney disease, lung disease or cardiac disease. The kidney disease may be chosen from the list comprising: focal segmental glomerulosclerosis (FSGS; including primary FSGS and secondary FSGS), fibrotic disorders in the kidney, chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension. The lung disease may be infectious or non-infectious inflammatory lung disease, preferably chosen from the list comprising: community acquired pneumonia, chronic obstructive pulmonary disease, asthma, bronchiectasis, bronchiolitis, bronchitis, emphysema, pleurisy or pulmonary fibrosis. The cardiac disease may be a coronary artery disease, preferably chosen from the list comprising: arthrosclerosis, pulmonary hypertension, ischaemic heart diseases, cardiomyopathies, and inflammatory heart diseases.

The endothelin A receptor inhibitor, CCR2 pathway inhibitor and/or the AT₁R blocker may be administered: in the same dosage form or in separate dosage forms. The endothelin A receptor inhibitor, CCR2 pathway inhibitor and the AT₁R blocker may be administered: concurrently or sequentially.

The endothelin A receptor inhibitor, CCR2 pathway inhibitor and/or the AT₁R blocker may be pharmaceutically acceptable salts of the endothelin A receptor inhibitor, CCR2 pathway inhibitor and/or the AT₁R blocker.

### Kits

The present invention provides a kit for the treatment, amelioration or prevention of a disease, said kit comprising:
a) at least one chemokine receptor 2 (CCR2) pathway inhibitor or a pharmaceutically acceptable salt thereof;
b) at least one endothelin A receptor inhibitor or a pharmaceutically acceptable salt thereof; and
c) instructions for use.

The present invention provides a kit for the treatment, amelioration or prevention of a disease, said kit comprising:
a) at least one chemokine receptor 2 (CCR2) pathway inhibitor or a pharmaceutically acceptable salt thereof;
b) at least one endothelin A receptor inhibitor or a pharmaceutically acceptable salt thereof;
c) at least one angiotensin type 1 receptor (AT₁R) blocker or a pharmaceutically acceptable salt thereof; and
d) instructions for use.

The contents of the kit can be lyophilized, and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of”′ have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### REFERENCES

Taylor et al, (2011) J Clin Hypertens. 13:677-686
Rios et al. (2000) Pharmacol. Ther. 92:71-87
Pfleger et al. (2006) Cell Signal 18:1664-1670
Pfleger et al. (2006) Nat Protoc 1:336-344
Ayoub et al. (2015) PLOS One 10(3):e0119803

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Assay for Receptor Functional Interaction

HEK293FT cells were seeded in 6-well plates at a density of approximately 700,000 cells/well and maintained at 37° C., 5% CO2 in Complete Media (DMEM containing 0.3 mg/ml glutamine, 100 IU/ml penicillin and 100 μg/ml streptomycin (Thermo Fisher)) supplemented with 10% fetal calf serum (FCS; Bovogen). Transient transfections were carried out 24 h after seeding using FuGene6 (Promega) according to manufacturer instructions. 24 h post-transfection, cells were washed with PBS, detached using 0.05% trypsin/0.53 mM EDTA, resuspended in phenol red free Complete Media containing 5% FCS and added to a poly-L-lysine-coated white 96-well microplate (Greiner Bio-One). 48 h post-transfection, bioluminescence resonance energy transfer (BRET) assays were carried out following pre-incubation of cells with EnduRen™ (Promega) at a final concentration of 30 μM, at 37° ° C., 5% CO₂ for 2 h. BRET measurements were taken at 37° C. using the CLARIOstar (BMG Labtech). Filtered light emissions were sequentially measured for 1 s in each of the ‘donor wavelength window’ (410-490 nm) and ‘acceptor wavelength window’ (520-620 nm).

For antagonist assays, cells were pre-incubated in EnduRen for 2 h. The antagonists (10 μM) were added and the assay was read for 30 min. Subsequently, the agonists (CXCL8 10 nM, AngII 100 nM) were added and the measurements continued for another 1 h.

The BRET signal observed between interacting proteins is normalized by subtracting the background BRET ratio. This can be done in one of two ways (see Pfleger et al. (2006) Cell Signal 18:1664-1670; Pfleger et al. (2006) Nat Protoc 1:336-344): 1) the ratio of the 520-550 nm emission over the 460-490 nm emission for a cell sample containing only the donor construct is subtracted from the same ratio for a sample containing the interacting acceptor and donor fusion proteins; 2) the ratio of the 520-550 nm emission over the 460-490 nm emission for a cell sample treated with vehicle is subtracted from the same ratio for a second aliquot of the same cell sample treated with ligand. In the following examples, the second calculation will be used and the signal is described as ‘ligand-induced BRET’.

The Receptor-Heteromer Investigation Technology (Receptor-HIT) is an assay configuration that provides insights into receptor complexes (Ayoub et al. (2015) PLOS One 10(3):e0119803). It is also known as GPCR-HIT when assessing GPCRs. It is an assay configuration whereby one receptor (eg. CCR2) is labelled with one component (eg. Renilla luciferase variant Rluc8) of a proximity-based reporter system (eg. BRET), the complementary component of which (eg. yellow fluorescent protein Venus) is fused to a receptor interacting partner (eg. β-arrestin 2). Treatment with a ligand (eg. AngII) selective for the receptor untagged with respect to the BRET assay (eg. hemagglutinin epitope-tagged AT₁R; HA-AT₁R) results in modulation of the proximity of the BRET-tagged receptor and the interacting partner, resulting in a change in BRET signal that is indicative of functional interaction between the two receptors.

### Results

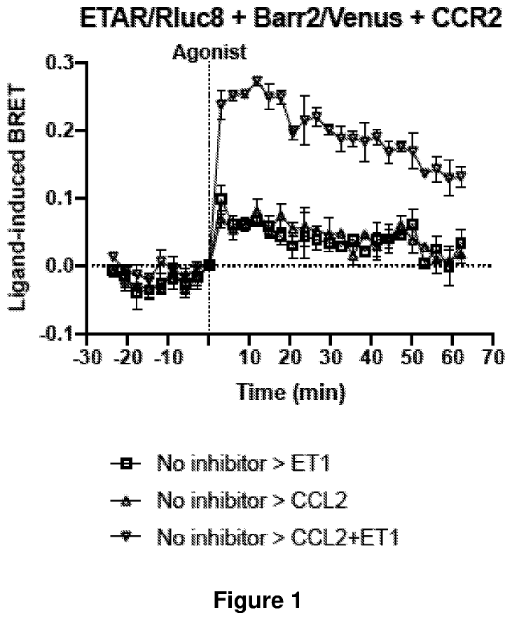

FIG. 1 is a graph of ETAR/Rluc8+Barr2/Venus+CCR2 which shows that when ETAR is tagged with Rluc8 and β-arrestin is tagged with Venus in the presence of unlabelled CCR2, β-arrestin is recruited to ETAR in the presence of ET-1 as expected, but also in the presence of CCL2/MCP1 which confirms the presence of a complex between receptors. The presence of both ligands causes hyperactivation of β-arrestin recruitment, confirming the interaction of the heteromer.

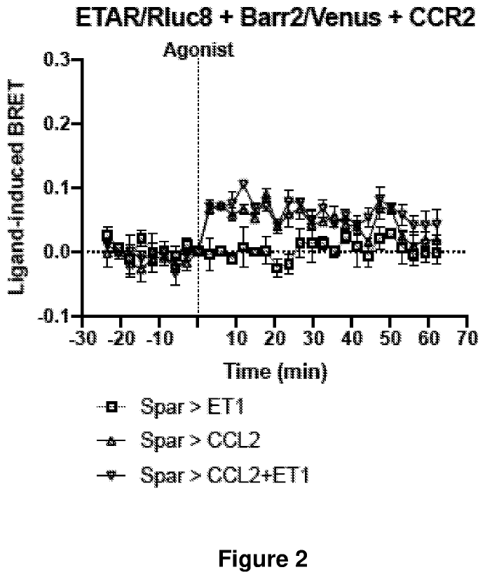

FIG. 2 is a graph of ETAR/Rluc8+Barr2/Venus+CCR2 which shows β-arrestin recruitment to ETAR occurs when CCL2 is present, confirming the interaction of ETAR and unlabelled CCR2. Sparsentan is able to abrogate the hyperactivation when both CCL2/CMP-1 and ET-1 ligands are present but not cease recruitment of β-arrestin to ETAR.

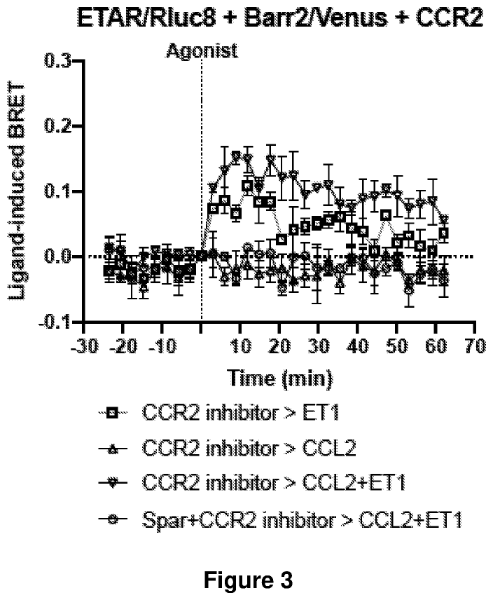

FIG. 3 is a graph of ETAR/Rluc8+Barr2/Venus+CCR2 which shows the hyperactivation of ETAR and CCR2 heteromer is abrogated by the addition of either sparsentan or CCR2 inhibition. ETAR is able to recruit β-arrestin at a low magnitude when stimulated with ET-1 regardless of the presence of CCR2, and sparsentan inhibits this signalling independent of CCR2 activation. Complete inhibition of β-arrestin recruitment to ETAR only occurs when ET-1 and CCL2/MCP-1 are present when CCR2 and ETAR are inhibited simultaneously.

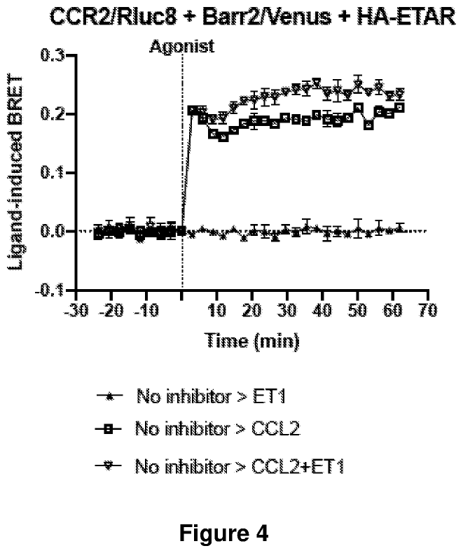

FIG. 4 is a graph of CCR2/Rluc8+Barr2/Venus+HA-ETAR which shows, in a different experimental orientation where CCR2 is tagged with Rluc8 and β-arrestin is tagged with Venus in the presence of unlabelled ETAR, β-arrestin is recruited to CCR2 in the presence of CCL2/MCP-1. Increased recruitment of β-arrestin is observed in the presence of both CCL2/MCP-1 and ET-1 ligands confirming the presence of an interaction between CCR2 and ETAR.

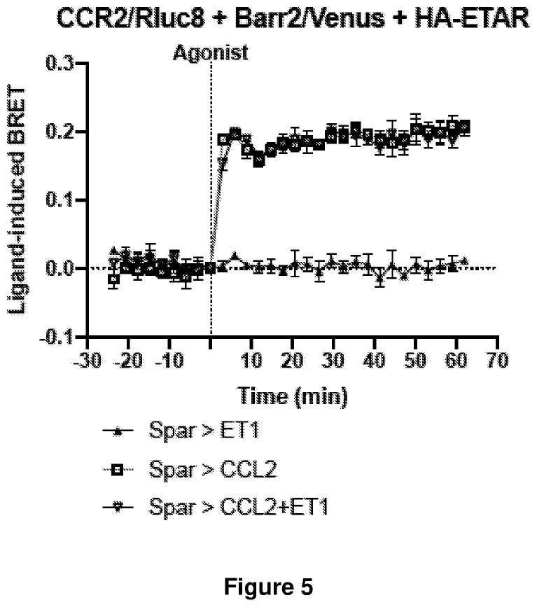

FIG. 5 is a graph of CCR2/Rluc8+Barr2/Venus+HA-ETAR which shows increased recruitment of β-arrestin is not observed in the presence of CCL2/MCP-1 and ET-1 when ETAR activation is blocked with sparsentan.

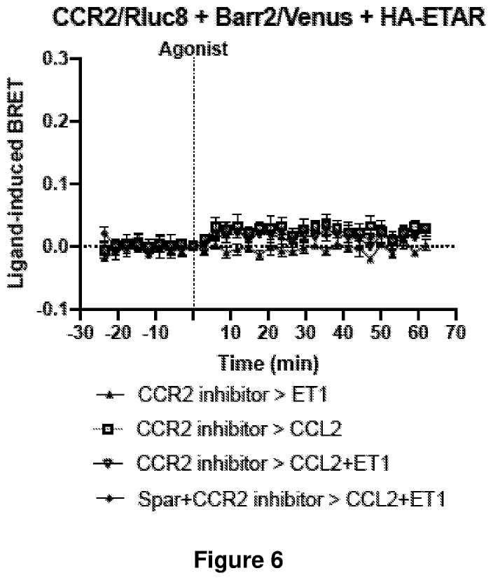

FIG. 6 is a graph of CCR2/Rluc8+Barr2/Venus+HA-ETAR which shows that there is no recruitment of β-arrestin to CCR2 in the presence of CCL2/MCP-1 when CCR2 is inhibited, regardless of the presence or absence of ETAR activation with ET-1.

### Example 2

### Formulations

Exemplary formulations for use in the present invention.

TABLE 1
Exemplary formulations

| Endothelin | | | Endothelin | | |
|---|---|---|---|---|---|
| A receptor | CCR2 pathway | AT₁R | A receptor | CCR2 pathway | AT₁R |
| inhibitors | inhibitor | blocker | inhibitors | inhibitor | blocker |
| Sparsentan | Repagermanium | | Sparsentan | Repagermanium | losartan |
| Sitaxentan | Repagermanium | | Sitaxentan | Repagermanium | losartan |
| Ambrisentan | Repagermanium | | Ambrisentan | Repagermanium | losartan |
| Atrasentan | Repagermanium | | Atrasentan | Repagermanium | losartan |
| BQ-123 | Repagermanium | | BQ-123 | Repagermanium | losartan |
| zibotentan | Repagermanium | | zibotentan | Repagermanium | losartan |
| bosentan | Repagermanium | | bosentan | Repagermanium | losartan |
| macitentan, | Repagermanium | | macitentan, | Repagermanium | losartan |
| tezosentan | Repagermanium | | tezosentan | Repagermanium | losartan |
| Sparsentan | Propagermanium | | Sparsentan | Propagermanium | losartan |
| Sitaxentan | Propagermanium | | Sitaxentan | Propagermanium | losartan |
| Ambrisentan | Propagermanium | | Ambrisentan | Propagermanium | losartan |
| Atrasentan | Propagermanium | | Atrasentan | Propagermanium | losartan |
| BQ-123 | Propagermanium | | BQ-123 | Propagermanium | losartan |
| zibotentan | Propagermanium | | zibotentan | Propagermanium | losartan |
| bosentan | Propagermanium | | bosentan | Propagermanium | losartan |
| macitentan, | Propagermanium | | macitentan, | Propagermanium | losartan |
| tezosentan | Propagermanium | | tezosentan | Propagermanium | losartan |
| Sparsentan | CCX140 | | Sparsentan | CCX140 | losartan |
| Sitaxentan | CCX140 | | Sitaxentan | CCX140 | losartan |
| Ambrisentan | CCX140 | | Ambrisentan | CCX140 | losartan |
| Atrasentan | CCX140 | | Atrasentan | CCX140 | losartan |
| BQ-123 | CCX140 | | BQ-123 | CCX140 | losartan |
| zibotentan | CCX140 | | zibotentan | CCX140 | losartan |
| bosentan | CCX140 | | bosentan | CCX140 | losartan |
| macitentan, | CCX140 | | macitentan, | CCX140 | losartan |
| tezosentan | CCX140 | | Sparsentan | Repagermanium | valsartan |
| Sparsentan | Repagermanium | irbesartan | Sitaxentan | Repagermanium | valsartan |
| Sitaxentan | Repagermanium | irbesartan | Ambrisentan | Repagermanium | valsartan |
| Ambrisentan | Repagermanium | irbesartan | Atrasentan | Repagermanium | valsartan |
| Atrasentan | Repagermanium | irbesartan | BQ-123 | Repagermanium | valsartan |
| BQ-123 | Repagermanium | irbesartan | zibotentan | Repagermanium | valsartan |
| zibotentan | Repagermanium | irbesartan | bosentan | Repagermanium | valsartan |
| bosentan | Repagermanium | irbesartan | macitentan, | Repagermanium | valsartan |
| macitentan, | Repagermanium | irbesartan | tezosentan | Repagermanium | valsartan |
| tezosentan | Repagermanium | irbesartan | Sparsentan | Propagermanium | valsartan |
| Sparsentan | Propagermanium | irbesartan | Sitaxentan | Propagermanium | valsartan |
| Sitaxentan | Propagermanium | irbesartan | Ambrisentan | Propagermanium | valsartan |
| Ambrisentan | Propagermanium | irbesartan | Atrasentan | Propagermanium | valsartan |
| Atrasentan | Propagermanium | irbesartan | BQ-123 | Propagermanium | valsartan |
| BQ-123 | Propagermanium | irbesartan | zibotentan | Propagermanium | valsartan |
| zibotentan | Propagermanium | irbesartan | bosentan | Propagermanium | valsartan |
| bosentan | Propagermanium | irbesartan | macitentan, | Propagermanium | valsartan |
| macitentan, | Propagermanium | irbesartan | tezosentan | Propagermanium | valsartan |
| tezosentan | Propagermanium | irbesartan | Sparsentan | CCX140 | valsartan |
| Sparsentan | CCX140 | irbesartan | Sitaxentan | CCX140 | valsartan |
| Sitaxentan | CCX140 | irbesartan | Ambrisentan | CCX140 | valsartan |
| Ambrisentan | CCX140 | irbesartan | Atrasentan | CCX140 | valsartan |
| Atrasentan | CCX140 | irbesartan | BQ-123 | CCX140 | valsartan |
| BQ-123 | CCX140 | irbesartan | zibotentan | CCX140 | valsartan |
| zibotentan | CCX140 | irbesartan | bosentan | CCX140 | valsartan |
| bosentan | CCX140 | irbesartan | macitentan, | CCX140 | valsartan |
| macitentan, | CCX140 | irbesartan | tezosentan | CCX140 | valsartan |
| tezosentan | CCX140 | irbesartan | Sparsentan | Repagermanium | telmisartan |
| Sparsentan | Repagermanium | candesartan | Sitaxentan | Repagermanium | telmisartan |
| Sitaxentan | Repagermanium | candesartan | Ambrisentan | Repagermanium | telmisartan |
| Ambrisentan | Repagermanium | candesartan | Atrasentan | Repagermanium | telmisartan |
| Atrasentan | Repagermanium | candesartan | BQ-123 | Repagermanium | telmisartan |
| BQ-123 | Repagermanium | candesartan | zibotentan | Repagermanium | telmisartan |
| zibotentan | Repagermanium | candesartan | bosentan | Repagermanium | telmisartan |
| bosentan | Repagermanium | candesartan | macitentan, | Repagermanium | telmisartan |
| macitentan, | Repagermanium | candesartan | tezosentan | Repagermanium | telmisartan |
| tezosentan | Repagermanium | candesartan | Sparsentan | Propagermanium | telmisartan |
| Sparsentan | Propagermanium | candesartan | Sitaxentan | Propagermanium | telmisartan |
| Sitaxentan | Propagermanium | candesartan | Ambrisentan | Propagermanium | telmisartan |
| Ambrisentan | Propagermanium | candesartan | Atrasentan | Propagermanium | telmisartan |
| Atrasentan | Propagermanium | candesartan | BQ-123 | Propagermanium | telmisartan |
| BQ-123 | Propagermanium | candesartan | zibotentan | Propagermanium | telmisartan |
| zibotentan | Propagermanium | candesartan | bosentan | Propagermanium | telmisartan |
| bosentan | Propagermanium | candesartan | macitentan, | Propagermanium | telmisartan |
| macitentan, | Propagermanium | candesartan | tezosentan | Propagermanium | telmisartan |
| tezosentan | Propagermanium | candesartan | Sparsentan | CCX140 | telmisartan |
| Sparsentan | CCX140 | candesartan | Sitaxentan | CCX140 | telmisartan |
| Sitaxentan | CCX140 | candesartan | Ambrisentan | CCX140 | telmisartan |
| Ambrisentan | CCX140 | candesartan | Atrasentan | CCX140 | telmisartan |
| Atrasentan | CCX140 | candesartan | BQ-123 | CCX140 | telmisartan |
| BQ-123 | CCX140 | candesartan | zibotentan | CCX140 | telmisartan |
| zibotentan | CCX140 | candesartan | bosentan | CCX140 | telmisartan |
| bosentan | CCX140 | candesartan | macitentan, | CCX140 | telmisartan |
| macitentan, | CCX140 | candesartan | tezosentan | CCX140 | telmisartan |
| tezosentan | CCX140 | candesartan | Sparsentan | Repagermanium | fimasartan |
| Sparsentan | Repagermanium | olmesartan | Sitaxentan | Repagermanium | fimasartan |
| Sitaxentan | Repagermanium | olmesartan | Ambrisentan | Repagermanium | fimasartan |
| Ambrisentan | Repagermanium | olmesartan | Atrasentan | Repagermanium | fimasartan |
| Atrasentan | Repagermanium | olmesartan | BQ-123 | Repagermanium | fimasartan |
| BQ-123 | Repagermanium | olmesartan | zibotentan | Repagermanium | fimasartan |
| zibotentan | Repagermanium | olmesartan | bosentan | Repagermanium | fimasartan |
| bosentan | Repagermanium | olmesartan | macitentan, | Repagermanium | fimasartan |
| macitentan, | Repagermanium | olmesartan | tezosentan | Repagermanium | fimasartan |
| tezosentan | Repagermanium | olmesartan | Sparsentan | Propagermanium | fimasartan |
| Sparsentan | Propagermanium | olmesartan | Sitaxentan | Propagermanium | fimasartan |
| Sitaxentan | Propagermanium | olmesartan | Ambrisentan | Propagermanium | fimasartan |
| Ambrisentan | Propagermanium | olmesartan | Atrasentan | Propagermanium | fimasartan |
| Atrasentan | Propagermanium | olmesartan | BQ-123 | Propagermanium | fimasartan |
| BQ-123 | Propagermanium | olmesartan | zibotentan | Propagermanium | fimasartan |
| zibotentan | Propagermanium | olmesartan | bosentan | Propagermanium | fimasartan |
| bosentan | Propagermanium | olmesartan | macitentan, | Propagermanium | fimasartan |
| macitentan, | Propagermanium | olmesartan | tezosentan | Propagermanium | fimasartan |
| tezosentan | Propagermanium | olmesartan | Sparsentan | CCX140 | fimasartan |
| Sparsentan | CCX140 | olmesartan | Sitaxentan | CCX140 | fimasartan |
| Sitaxentan | CCX140 | olmesartan | Ambrisentan | CCX140 | fimasartan |
| Ambrisentan | CCX140 | olmesartan | Atrasentan | CCX140 | fimasartan |
| Atrasentan | CCX140 | olmesartan | BQ-123 | CCX140 | fimasartan |
| BQ-123 | CCX140 | olmesartan | zibotentan | CCX140 | fimasartan |
| zibotentan | CCX140 | olmesartan | bosentan | CCX140 | fimasartan |
| bosentan | CCX140 | olmesartan | macitentan, | CCX140 | fimasartan |
| macitentan, | CCX140 | olmesartan | tezosentan | CCX140 | fimasartan |
| tezosentan | CCX140 | olmesartan | Sparsentan | Repagermanium | azilsartan |
| Sparsentan | Repagermanium | eprosartan | Sitaxentan | Repagermanium | azilsartan |
| Sitaxentan | Repagermanium | eprosartan | Ambrisentan | Repagermanium | azilsartan |
| Ambrisentan | Repagermanium | eprosartan | Atrasentan | Repagermanium | azilsartan |
| Atrasentan | Repagermanium | eprosartan | BQ-123 | Repagermanium | azilsartan |
| BQ-123 | Repagermanium | eprosartan | zibotentan | Repagermanium | azilsartan |
| zibotentan | Repagermanium | eprosartan | bosentan | Repagermanium | azilsartan |
| bosentan | Repagermanium | eprosartan | macitentan, | Repagermanium | azilsartan |
| macitentan, | Repagermanium | eprosartan | tezosentan | Repagermanium | azilsartan |
| tezosentan | Repagermanium | eprosartan | Sparsentan | Propagermanium | azilsartan |
| Sparsentan | Propagermanium | eprosartan | Sitaxentan | Propagermanium | azilsartan |
| Sitaxentan | Propagermanium | eprosartan | Ambrisentan | Propagermanium | azilsartan |
| Ambrisentan | Propagermanium | eprosartan | Atrasentan | Propagermanium | azilsartan |
| Atrasentan | Propagermanium | eprosartan | BQ-123 | Propagermanium | azilsartan |
| BQ-123 | Propagermanium | eprosartan | zibotentan | Propagermanium | azilsartan |
| zibotentan | Propagermanium | eprosartan | bosentan | Propagermanium | azilsartan |
| bosentan | Propagermanium | eprosartan | macitentan, | Propagermanium | azilsartan |
| macitentan, | Propagermanium | eprosartan | tezosentan | Propagermanium | azilsartan |
| tezosentan | Propagermanium | eprosartan | Sparsentan | CCX140 | azilsartan |
| Sparsentan | CCX140 | eprosartan | Sitaxentan | CCX140 | azilsartan |
| Sitaxentan | CCX140 | eprosartan | Ambrisentan | CCX140 | azilsartan |
| Ambrisentan | CCX140 | eprosartan | Atrasentan | CCX140 | azilsartan |
| Atrasentan | CCX140 | eprosartan | BQ-123 | CCX140 | azilsartan |
| BQ-123 | CCX140 | eprosartan | zibotentan | CCX140 | azilsartan |
| zibotentan | CCX140 | eprosartan | bosentan | CCX140 | azilsartan |
| bosentan | CCX140 | eprosartan | macitentan, | CCX140 | azilsartan |
| macitentan, | CCX140 | eprosartan | tezosentan | CCX140 | azilsartan |
| tezosentan | CCX140 | eprosartan | tezosentan | CCX140 | losartan |
| Sitaxentan | Repagermanium | Sparsentan | Sitaxentan | Propagermanium | Sparsentan |
| Ambrisentan | Repagermanium | Sparsentan | Ambrisentan | Propagermanium | Sparsentan |
| Atrasentan | Repagermanium | Sparsentan | Atrasentan | Propagermanium | Sparsentan |
| BQ-123 | Repagermanium | Sparsentan | BQ-123 | Propagermanium | Sparsentan |
| zibotentan | Repagermanium | Sparsentan | zibotentan | Propagermanium | Sparsentan |
| bosentan | Repagermanium | Sparsentan | bosentan | Propagermanium | Sparsentan |
| macitentan, | Repagermanium | Sparsentan | macitentan, | Propagermanium | Sparsentan |
| tezosentan | Repagermanium | Sparsentan | tezosentan | Propagermanium | Sparsentan |
| Sitaxentan | CCX140 | Sparsentan | zibotentan | CCX140 | Sparsentan |
| Ambrisentan | CCX140 | Sparsentan | bosentan | CCX140 | Sparsentan |
| Atrasentan | CCX140 | Sparsentan | macitentan, | CCX140 | Sparsentan |
| BQ-123 | CCX140 | Sparsentan | tezosentan | CCX140 | Sparsentan |

1. A pharmaceutical composition comprising:
a) at least one endothelin A receptor inhibitor or a pharmaceutically acceptable salt thereof; and
b) at least one chemokine receptor 2 (CCR2) inhibitor or a pharmaceutically acceptable salt thereof.

2. The pharmaceutical composition of claim 1 further comprising at least one an angiotensin type 1 receptor (AT₁R) blocker or a pharmaceutically acceptable salt thereof.

3. The pharmaceutical composition of claim 1 wherein the endothelin A receptor inhibitor is chosen from the group comprising: endothelin A receptor antagonists; dual endothelin A and B antagonists; and dual heterologous receptor antagonists and combinations thereof.

4. The pharmaceutical composition of claim 3 wherein:
a) the endothelin A receptor antagonist is chosen from the group comprising: sitaxentan, ambrisentan, atrasentan, BQ-123, zibotentan;
b) the dual endothelin A and B antagonist is chosen from the group comprising: bosentan, macitentan, tezosentan; and/or
c) the dual heterologous receptor antagonist is chosen from the group comprising: sparsentan.

5. The pharmaceutical composition of claim 1 wherein the chemokine receptor 2 (CCR2) inhibitor is chosen from the group comprising: repagermanium; propagermanium; CCX140; Cenicriviroc; PF-6309; PF-04178903; CCX872-B; MLN1202; and PF-04634817.

6. The pharmaceutical composition of claim 2 wherein the angiotensin type 1 receptor (AT₁R) blocker is chosen from the group comprising: sparsentan, irbesartan, eprosartan, losartan, valsartan, telmisartan, candesartan, olmesartan, ZD-7115, fimasartan, azilsartan, DuP 753, EXP 3174, embusartan, KRH-594, fonsartan and pratosartan.

7. The pharmaceutical composition of claim 1 or 2 wherein the CCR2 pathway inhibitor and endothelin A receptor inhibitor are administered in the same dosage form or in separate dosage forms.

8. The pharmaceutical composition of claim 1 wherein the CCR2 pathway inhibitor and endothelin A receptor inhibitor are administered concurrently or sequentially.

9. The pharmaceutical composition of claim 2 wherein the CCR2 pathway inhibitor, endothelin A receptor inhibitor and AT1R blocker are administered in the same dosage form or in separate dosage forms.

10. The pharmaceutical composition of claim 2 wherein the CCR2 pathway inhibitor, endothelin A receptor inhibitor and AT1R blocker are administered concurrently or sequentially.

11. A method for the treatment, amelioration or prevention of a disease comprising administering to a subject a therapeutically effective amount of a combination of:
a) at least one endothelin A receptor inhibitor or a pharmaceutically acceptable salt thereof; and
b) at least one chemokine receptor 2 (CCR2) inhibitor or a pharmaceutically acceptable salt thereof.

12. The method of claim 11 further comprising administering to the subject a therapeutically effective amount of at least one angiotensin type 1 receptor (AT1R) blocker or a pharmaceutically acceptable salt thereof.

13. The method of claim 11 wherein the disease is a kidney disease, lung disease or cardiac disease.

14. The method of claim 13 wherein:
the kidney disease is chosen from the list comprising: focal segmental glomerulosclerosis (FSGS; including primary FSGS and secondary FSGS), fibrotic disorders in the kidney, chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension;
the lung disease is an infectious or non-infectious inflammatory lung disease; and/or
the cardiac disease is a coronary artery disease.

15. The method of claim 14 wherein the infectious or non-infectious inflammatory lung disease is chosen from the list comprising: community acquired pneumonia, chronic obstructive pulmonary disease, asthma, bronchiectasis, bronchiolitis, bronchitis, emphysema, pleurisy or pulmonary fibrosis.

16. The method of claim 14 wherein the coronary artery disease is chosen from the list comprising: arthrosclerosis, pulmonary hypertension, ischaemic heart diseases, cardiomyopathies, and inflammatory heart diseases

17. Use of a pharmaceutical composition comprising:
a) at least one endothelin A receptor inhibitor or a pharmaceutically acceptable salt thereof; and
b) at least one chemokine receptor 2 (CCR2) inhibitor or a pharmaceutically acceptable salt thereof for the manufacture of a dosage form for the treatment, amelioration or prevention of a disease.

18. The use of claim 17 wherein the composition further comprises at least one an angiotensin type 1 receptor (AT₁R) blocker or a pharmaceutically acceptable salt thereof.

19. At least one endothelin A receptor inhibitor or a pharmaceutically acceptable salt thereof and at least one chemokine receptor 2 (CCR2) inhibitor or a pharmaceutically acceptable salt thereof, for use in a formulation for the treatment, amelioration or prevention of a disease.

20. At least one endothelin A receptor inhibitor or a pharmaceutically acceptable salt thereof, at least one chemokine receptor 2 (CCR2) inhibitor or a pharmaceutically acceptable salt thereof, and at least one angiotensin type 1 receptor (AT₁R) blocker or a pharmaceutically acceptable salt thereof for use in a formulation for the treatment, amelioration or prevention of a disease.

21. At least one endothelin A receptor inhibitor for use in a formulation for the treatment, amelioration or prevention of a disease, wherein the at least one endothelin A receptor inhibitor is administered to the subject concurrently or sequentially with at least one CCR2 pathway inhibitor.

22. At least one CCR2 pathway inhibitor for use in a formulation for the treatment, amelioration or prevention of a disease wherein the at least one CCR2 pathway inhibitor is administered to the subject concurrently or sequentially with at least one endothelin A receptor inhibitor.

23. At least one endothelin A receptor inhibitor for use in a formulation for the treatment, amelioration or prevention of a disease, wherein the at least one endothelin A receptor inhibitor is administered to the subject concurrently or sequentially with at least one CCR2 pathway inhibitor and at least one angiotensin type 1 receptor blocker.

24. At least one CCR2 pathway inhibitor for use in a formulation for the treatment, amelioration or prevention of a disease wherein the at least one CCR2 pathway inhibitor is administered to the subject concurrently or sequentially with at least one endothelin A receptor inhibitor and at least one angiotensin type 1 receptor blocker.

25. At least one angiotensin type 1 receptor blocker for use in a formulation for the treatment, amelioration or prevention of a disease wherein the at least one angiotensin type 1 receptor blocker is administered to the subject concurrently or sequentially with at least one endothelin A receptor inhibitor and at least one CCR2 pathway inhibitor.

26. A kit for the treatment, amelioration or prevention of a disease, said kit comprising:
a) at least one chemokine receptor 2 (CCR2) pathway inhibitor or a pharmaceutically acceptable salt thereof;
b) at least one endothelin A receptor inhibitor or a pharmaceutically acceptable salt thereof; and
c) instructions for use.

27. The kit of claim 26 further comprising at least one angiotensin type 1 receptor (AT1R) blocker or a pharmaceutically acceptable salt thereof.
